(19) United States
(12) Patent Application Publication
(10) Pub. No.: US 2025/0222012 A1
(43) Pub. Date: Jul. 10, 2025

(54) USE OF CANNABINOID COMPOSITIONS FOR TREATING PAIN ASSOCIATED WITH SMALL FIBER NEUROPATHY

(71) Applicant: Schedule 1 Therapeutics, Inc., Chicago, IL (US)

(72) Inventors: George D. Pappas, Chicago, IL (US); Gregory Dussor, Chicago, IL (US)

(21) Appl. No.: 18/876,110

(22) PCT Filed: Nov. 2, 2022
(86) PCT No.: PCT/US2022/048674

§ 371 Date: Dec. 17, 2024

Related U.S. Application Data
(60) Provisional application No. 63/274,744, filed on Nov. 2, 2021.

(51) Int. Cl. A61K 31/00

(52) U.S. Cl. CPC A61K 31/658

(57) ABSTRACT

The present disclosure is related to the use of compositions and their use in reducing pain associated with small fiber neuropathies, and in particular chemotherapy induced peripheral neuropathy.

### CROSS REFERENCE OF RELATED APPLICATION

This application claims priority to U.S. Provisional Application No. 63/274,744 entitled “USE OF CANNABINOID COMPOSITIONS FOR TREATING PAIN ASSOCIATED WITH SMALL FIBER NEUROPATHY” filed Nov. 2, 2021, the entirety of which is incorporated by reference herein.

### FIELD OF THE DISCLOSURE

The present disclosure is related to the use of compositions comprising cannabinoids and their use in reducing pain associated with small fiber neuropathy, including such pain associated with chemotherapy.

### BACKGROUND OF THE DISCLOSURE

In the peripheral nervous system, diseases can affect all types of nerves, or can be confined to only parts of the nerve, for instance just the myelin or axon. Similarly, a disease can affect just large fiber neurons or small fiber neurons. Diseases that affect purely small fibers can present as purely sensory disruption such as pain, purely autonomic dysfunction, or a combination thereof. Categorizing small fiber neuropathy (SFN) into different subsets of neuropathies according to clinical phenotypes allows for a better understanding of the pathophysiology, a more precise diagnosis, and improved potential treatments.

Small fiber neuropathy is the result of damage to peripheral nerves, including those that are small and myelinated (Aδ), as well as those that are unmyelinated (unmyelinated C fibers). In SFN, small somatic and autonomic fibers can be affected. Normally, these fibers control thermal and pain perception and control autonomic and enteric functions. Thus, patients with SFN can present with either autonomic or somatic symptoms, or both. Symptoms can include allodynia, burning, lower thermal sensation, hyperesthesia, paresthesia, numbness in the lower extremities with potential to affect limbs and trunk, restless leg syndrome, dry eyes and mouth, abnormal sweating, bladder control issues, gastric issues, skin discoloration, cardiac symptoms, and others. Cardiac symptoms include syncope, palpitations, and orthostatic hypotension. Small fiber neuropathy has a poorly understood pathology. It can be a result of a variety of diseases, including diabetes mellitus, autoimmune disorders such as Sjögren or sarcoidosis, paraproteinemia, and paraneoplastic syndrome, with diabetes mellitus being the most common cause of SFN. Hereditary amyloid neuropathy also results in damage to small nerve fibers. Amyloid neuropathies can be multisystemic or relegated to the cardiac system or only neuropathy. There can be some presentation of neuropathy and cardiac symptoms without being widespread. Familial amyloid neuropathies include those caused by mutations in transthyretin (TTR) amyloidosis, apoprotein A1, and gelsolin.

Syndromes with small fiber pathology may include diabetes mellitus (i.e. diabetic polyneuropathy), alcohol-related neuropathy, autoimmune diseases, chemotherapy induced SFN, hereditary SFN, neurodegenerative diseases (e.g, for example, Parkinson's and Amyotrophic Lateral Sclerosis), and others.

The underlying cause of the particular type of SFN should be addressed to treat the SFN (for example, glucose control, exercise for dysglycemia-associated SFN, or treatments intending to address small fiber degeneration or nociceptor sensitization, abnormal signal transmission, ectopic generation of action potentials, disturbance in synaptic communication, or formation of pathologic neural circuitry, and others). Treatments to address neuropathic pain include the use of FDA-approved therapies, such as tricyclic antidepressants (TCAs) serotonin-norepinephrine reuptake inhibitors (SNRIs), anticonvulsants including gabapentin and pregabalin, opioids, and non-FDA approved alternative treatments, such as cannabinoids. Topical treatments that contain drugs such as lidocaine, capsaicin, and other treatments such as those targeting transient receptor potential channels, angiotensin II type 2 receptor (ATR2) antagonism, intrathecal delivery of medications to reduce systemic exposure, and use of erythropoietin (EPO). Intravenous immunoglobulin G, anti-tumor necrosis factor, or a combination, and small peptides derived from EPO are used to treat immune-mediated, sarcoidosis-associated SFNs. Intravenous immunoglobulin (IVIg) is to treat patients with idiopathic SFN. Inherited amyloid polyneuropathies are treated with conventional neuropathy drugs or surgical intervention such as liver transplantation for familial amyloid polyneuropathy (FAP). Tafamidis is used in symptomatic Transthyretin-Related Familial Amyloid Polyneuropathy (TTR-FAP), as is diflunisal, a nonsteroidal anti-inflammatory drug (NSAID), and combination of doxycycline and tauroursodeoxycholic acid. Short synthetic oligonucleotides (ASOs) and small-interfering RNAs (RNAi) monoclonal antibodies are used to effect disease progression.

Chemotherapy-induced peripheral neuropathy (CIPN) is a common outcome of treatment with multiple classes of chemotherapeutic agents including taxanes (e.g. paclitaxel), platinums (e.g. oxaliplatin, cisplatin), vinka alkaloids (e.g. vincristine), and bortezomib. CIPN is present in over 50% of patients treated with chemotherapy and symptoms include those commonly associated with peripheral neuropathies such as extreme pain (spontaneous and evoked), paresthesia, or loss of sensation. The presence of CIPN is often dose limiting for the chemotherapeutic agent i.e. doses needed for full efficacy against tumors cannot be achieved due to the presence of neuropathies. While in some cases CIPN symptoms resolve after discontinuation of the chemotherapeutic agent, many patients have residual symptoms of CIPN even years after treatment. While the exact mechanism causing CIPN remain unclear, there is thought to be damage to peripheral sensory neurons, particularly those that have long nerve fibers such as in the hands and feet. CIPN commonly presents as pain in a “stocking and glove” pattern where symptoms are present along the legs and arms, into the feet and hands, respectively.

Treatment of chemotherapy pain includes the use of FDA-approved therapies, and non-FDA approved alternative treatments, such as cannabinoids. For instance, current treatments may include, NSAIDs, opioids, simple analgesics, barbiturates, anti-epileptic drugs, tricyclic anti-depressants, COX-2 inhibitors, and others.

Such treatments, however, often fail to alleviate pain associated with chemotherapy. Hence, there is a need in the art for an effective treatment of pain associated with chemotherapy.

### SUMMARY OF THE INVENTION

Provided herein is a method of reducing pain associated with a small fiber neuropathy in a subject in need thereof. The method may comprise administering to the subject a composition comprising a pharmaceutically effective amount of one or more minor cannabinoids, which may be isolated. The one or more minor cannabinoids may be administered alone or in combination with one or more of a tetrahydrocannabinol (THC) and a cannabidiol (CBD). The method may modulate at least one biomarker of central and peripheral sensitization in the subject.

The one or more minor cannabinoids may be selected from the group consisting of cannabigerovarin (CBG), cannbigerolic acid (CBGA), cannabinol (CBN), cannabinolic acid (CBNA), tetrahydrocannabivarin (THCV), tetrahydrocannabinolic acid (THCA), cannabichromene (CBC), cannabichromenic acid (CBCA), cannabivarin (CBDV), and delta-8-tetrahydrocannabinol (D8-THC).

The composition may comprise a ratio of the total amount of the one or more minor cannabinoids to the amount of THC. The ratio may maximize the modulation of at least one biomarker of central and peripheral sensitization. The composition may comprise CBD.

The composition may comprise a ratio of the total amount of the one or more minor cannabinoids to the amount of CBD. The ratio may maximize the modulation of at least one biomarker of central and peripheral sensitization. The composition may comprise THC.

The composition may comprise THC and CBD. The composition may further comprise one or more of a terpenoid and an additional analgesic agent.

Also provided herein is a method of reducing pain associated with a small fiber neuropathy in a subject in need thereof, comprising administering to the subject a composition comprising a pharmaceutically effective amount of THC and CBD. The method may modulate at least one biomarker of central and peripheral sensitization in the subject, while reducing undesired side effects or risk factors of THC.

The composition may comprise a ratio of the amount CBD to the amount of THC (a CBD: THC ratio). The ratio may maximize the modulation of at least one biomarker of central and peripheral sensitization while reducing undesired side effects or risk factors of THC. The CBD: THC ratio may be from 30:1 to 300:1, and may be 100:1 or 30:1.

The composition may further comprise a minor cannabinoid selected from the group consisting of CBG, CBGA, CBN, CBNA, THCV, THCA, CBC, CBCA, CBDV, and D8-THC. The composition may further comprise one or more of a terpenoid and an additional analgesic agent.

The small fiber neuropathy in the methods provided herein may be a chemotherapy-induced peripheral neuropathy. The pain associated with small fiber neuropathy may be chemotherapy pain. The chemotherapy may be one or more of a taxane, paclitaxel, a platinum derivative, oxaliplatin, cisplatin, a vinka alkaloid, vincristine, and bortezomib.

The at least one biomarker modulated in the methods provided herein may be selected from the group consisting of NMDA, glutamate, CGRP, FAAH, Substance P, 5-HT, NO, GABA, NGF, serotonin, dopamine, AEA, 2-AG, BDNF, microRNA, NF-L, NF-H, OPN, glycerophospholipids, 1-deoxysphingolipids, 2-hydroxybutyrate, 3-hydroxybutyrate, pyruvate, o-acetyl-carnitine, sphingolipids, glutathione, lysine, and cholesteryl esters lyso-phosphatidylcholines.

### DETAILED DESCRIPTION

The mechanisms of action of cannabinoids for CIPN or for SFNs are not clear. Cannabinoids are known to inhibit neuronal excitability, to decrease inflammatory responses, and to modulate a list of ion channels and receptors, any of which could contribute to efficacy for CIPN and for other SFNs.

The present disclosure details compositions comprising at least one cannabinoid. For instance, in some embodiments, the present disclosure details methods of using compositions comprising at least one minor cannabinoid for pain associated with chemotherapy.

In additional aspects, the present disclosure details compositions comprising tetrahydrocannabinol (THC) and/or cannabidiol (CBD). Specifically, a composition of the disclosure may comprise THC and an amount of CBD to reduce any unwanted side effects of THC or risk factors associated with THC, or to increase efficacy of THC or of the composition. Non-limiting examples of side effects or risk factors include psychoactivity, psychosis, anxiety, paranoia, dizziness, confusion, cardiovascular risk, memory impairment, or combinations thereof. Additionally, the present disclosure details methods of using such compositions for pain associated with chemotherapy.

### I. Minor Cannabinoid Compositions

Compositions of cannabinoids to treat SFNs and in particular CIPN consist of CBD and THC, with higher doses of the former. Dosing strategies for SFNs and in particular CIPN may include either preventative or therapeutic strategies. For preventative dosing, cannabinoids may be given prior to or during treatment with chemotherapeutic agents to prevent the development of SFN or of CIPN. For therapeutic dosing, cannabinoids may be given either during treatment with chemotherapeutic agents, after SFN or CIPN symptoms have developed, or following termination of treatment with chemotherapeutic agents, when SFN or CIPN symptoms still persist.

A minor cannabinoid composition may comprise at least one minor cannabinoid. In some embodiments, the composition may further comprise a major cannabinoid, a terpenoid, an additional compound, or a combination thereof.

The minor cannabinoid composition, when administered to a subject in need thereof, may modulate one or more biomarkers of central and peripheral sensitization. As used herein, the phrase peripheral sensitization may refer to hypersensitivity in peripheral nociceptors which cause phenomena such as reduced stimulus threshold (allodynia), increase in response and prolonged after effects (hyperalgesia), and expansion of the receptive field to non-injured tissue. As used herein, “central sensitization” may refer to the hyperexcitable activity of central nociceptive neural pathways.

The minor cannabinoid composition may modulate biomarkers of central and peripheral sensitization by binding as an agonist, antagonist, or inverse agonist to one or more receptors from the group consisting of CB1, CB2 (in the dorsal horn, spinal cord), transient receptor potential vanilloid (TRPV)-1, TRPV-2, TRPV-3, TRPV-4, TRPV-8, TRPA-1, TRPM-8, 5-hydroxytryptamine 5HT-1A, 5HT-2A, Type 3 5HT, alpha-2 adrenoceptor, PPAR-gamma, GAPDH, PPAR, GPCR-92 NK1, EP, trkB,, mGlu1, mGlu4, mGlu5 mGlu6, mGlu7, mGlu8, FAAH, NMDA and its subunits NR1 and NR2A.

The minor cannabinoid composition may modulate 5-HT receptors (including 5HT-1B/1D receptors), suppress glutamatergic release via CB1 receptor mediated inhibition of NMDA, activate TRPV receptors to modulate CGRP release and influence vasomotor tone, increase AEA levels in cerebrospinal fluid and in the PAG, or decrease CGRP and NO. In one example, the minor cannabinoid composition modulates biomarkers of central and peripheral sensitization by binding as an agonist, antagonist, or inverse agonist to one or more receptors from the group consisting of CB1, CB2, TRPV1, TRPV2, NMDA and glutamate receptors.

Biomarkers of central and peripheral sensitization include NMDA, CGRP, FAAH, Substance P, Glutamate, NO, GABA, NGF, serotonin, dopamine, AEA, 2-AG, BDNF, microRNA, NF-L, NF-H, OPN, glycerophospholipids, 1-deoxysphingolipids, 2-hydroxybutyrate, 3-hydroxybutyrate, pyruvate, o-acetyl-carnitine, sphingolipids, glutathione, lysine, and cholesteryl esters lyso-phosphatidylcholines. In some embodiments, biomarkers of peripheral sensitization include one or more of neurogenic CGRP and nitric oxide.

In certain embodiments, the minor cannabinoid composition decreases the activity of enzymes that act as biomarkers, such as cyclooxygenase, lipoxygenase and P450-type enzymes such as CYP1A1, CYP1A2, CYP2B6, CYP2C9, CYP3A4, CYP3A5, CYP2A6, CYP2D6, CYP1B1, and/or CYP3A7. Decreasing the activity of one or more of these enzymes may modulate the anti-inflammatory effects that are implicated in chemotherapy pain.

In particular embodiments, the minor cannabinoid composition exhibits other forms of anti-nociception by inhibiting NF-KB activity, iNOS, corticotropin releasing factor binding protein, cysteine protease ATGB4, photoreceptor-specific nuclear receptor NR2E3, or diacylglycerol lipase. The minor cannabinoid composition may modulate levels of NMDA, alpha amino-3-hydroxyl-5-methyl-4-isoxazole-proprionate, and kainate receptor modulated neuro-toxicities via CB1, or suppress glutamatergic release by inhibiting modulation of NMDA, mediated by CB1, or increase endo-opioid pre-curser gene expression involved in pain perception. In one example, the minor cannabinoid composition affects sensitization by modulating levels of NMDA and/or glutamate.

### (a) Minor Cannabinoids

The minor cannabinoid composition may comprise at least one minor cannabinoid. The one or more minor cannabinoids may be isolated from natural sources or synthetically manufactured. Non-limiting examples of minor cannabinoids include CBGV (cannabigerovarin), CBGVA (cannabigerovarinic acid), CBG (cannabigerol), CBGA (cannbigerolic acid), Cannabinerolic acid, Cannabinerol, CBN (cannabinol), CBNA (cannabinolic acid), THCV (tetrahydrocannabivarin), THCA (tetrahydrocannabinolic acid), D8-THC (delta-8-tetrahydrocannabinol), CBDA (cannabidiolic acid), CBC (cannabichromene), CBCA, (cannabichromenic acid), CBDV (cannabivarin), CBL (Cannabicyclol), CBLA (Cannabicyclolic acid), Cannabicyclovarin, CBE (cannabielsoin), CBF (cannabifuran), Cannabicitran, Cannabitriol, and Cannabiorcol. In one embodiment, the minor cannabinoid composition comprises one or more of CBGV (cannabigerovarin), CBGVA (cannabigerovarinic acid), CBG (cannabigerol), CBGA (cannbigerolic acid), Cannabinerolic acid, Cannabinerol, CBN (cannabinol), CBNA (cannabinolic acid), THCV (tetrahydrocannabivarin), THCA (tetrahydrocannabinolic acid), D8-THC (delta-8-tetrahydrocannabinol), CBDA (cannabidiolic acid), CBC (cannabichromene), CBCA, (cannabichromenic acid), CBDV (cannabivarin), CBL (Cannabicyclol), CBLA (Cannabicyclolic acid), Cannabicyclovarin, CBE (cannabielsoin), CBF (cannabifuran), Cannabicitran, Cannabitriol, and Cannabiorcol. In another embodiment, the minor cannabinoid composition of comprises two or more, three or more, four or more, five or more, six or more, seven or more, eight or more, nine or more, or ten or more of the minor cannabinoids. In yet another embodiment, the minor cannabinoid composition comprises at least 11, at least 12, at least 13, at least 14, at least 15, at least 16, at least 17, at least 18, at least 19, at least 20, at least 21 or at least 22 or more of the minor cannabinoids.

In some embodiments, the at least one minor cannabinoid is selected from the group consisting of CBG, CBGA, CBN, CBNA, THCV, THCA, CBC, CBCA, CBDV, and D8-THC. In certain embodiments, the minor cannabinoid composition comprises at least two, three, four, five, six, seven, eight, nine or ten of the minor cannabinoid(s). The minor cannabinoid composition may comprise any combination of CBG, CBGA, CBN, CBNA, THCV, THCA, CBC, CBCA, CBDV, and D8-THC.

The minor cannabinoid composition may comprise from about 1% to about 100% of one or more minor cannabinoids. For instance, the minor cannabinoid composition may comprise about 5, 10, 15, 20, 25, 30, 35, 40, 45, 50, 55, 60, 65, 70, 75, 80, 85, 90, or 95% by weight of the one or more minor cannabinoids. In some embodiments, the minor cannabinoid composition may comprise from about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 100% of the one or more minor cannabinoids.

### (b) Major Cannabinoids

The minor cannabinoid composition may comprise a major cannabinoid. Non-limiting examples of a major cannabinoid include THC (also referred to as Delta-9-THC) and CBD. In one embodiment, the minor cannabinoid composition comprises THC. In another embodiment, the minor cannabinoid composition comprises CBD. In a further example, the minor cannabinoid composition comprises both THC and CBD.

The minor cannabinoid composition may comprise from about 1% to about 99% by weight of the one or more major cannabinoids. For instance, the composition may comprise about 5, 10, 15, 20, 25, 30, 35, 40, 45, 50, 55, 60, 65, 70, 75, 80, 85, 90, or 95% of the one or more major cannabinoids. In some embodiments, the minor cannabinoid composition comprises about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 99% of the one or more major cannabinoids.

### (c) Terpenoids

In certain embodiments, the minor cannabinoid composition comprises one or more terpenoids. Non-limiting examples of terpenoids include myrcene, β-caryophyllene, limonene, α-terpineol, linalool, α-phellandrene, α-pinene, β-pinene, γ-terpinene, nerolidol, phytol, caryophyllene oxide, and α-humule. In some embodiments, the minor cannabinoid composition comprises one, two, three, four, five, or more than five of the terpenoids.

The minor cannabinoid composition may comprise from 0% to about 99% by weight of the at least one terpenoid. For instance, the minor cannabinoid composition may comprise about 5, 10, 15, 20, 25, 30, 35, 40, 45, 50, 55, 60, 65, 70, 75, 80, 85, 90, or 95% of the at least one terpenoid. In some embodiments, the minor cannabinoid composition comprises from about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 99% of the at least one terpenoid.

### (d) Other Compounds

In certain embodiments, the minor cannabinoid composition comprises one or more additional compounds. Non-limiting examples of additional compounds include a simple analgesic like paracetamol and/or acetaminophen, a non-steroidal anti-inflammatory drug (NSAID), a 5-HT (serotonin) receptor agonist, a calcitonin gene related peptide (CGRP) receptor antagonist, an N-methyl D-asparate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, and a NO inhibitor. In some embodiments, the minor cannabinoid composition comprises one, two, three, four, five, or more than five of the additional compounds.

The minor cannabinoid composition may comprise from 0% to about 99% by weight of the at least one additional compound. For instance, the minor cannabinoid composition may comprise about 5, 10, 15, 20, 25, 30, 35, 40, 45, 50, 55, 60, 65, 70, 75, 80, 85, 90, or 95% of one or more additional compounds. In some embodiments, a minor cannabinoid composition may comprise from about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 99% of one or more additional compounds.

### (e) Amounts

The minor cannabinoid composition of may be used to deliver about 20, 30, 40, 50, 60, 70, 80, 90, 100, 110, 120, 130, 140, 150, 160, 170, 180, or more than 180 mg/day of THC. In certain embodiments, up to 40 mg THC is delivered per day. In other embodiments, up to about 120 mg THC is delivered per day. These amounts may be contained in a single dose, or more than one dose (e.g. two, three, or more than three doses).

The minor cannabinoid composition may be used to deliver about 100, 200, 300, 400, 500, 600, 700, 800, 900, 1000, 1100, 1200, 1300, 1400, 1500, 1600, 1700, 1800, 1900, 2000, 2100, 2200, 2300, or 2400 mg/day of CBD. These amounts may be contained in a single dose, or more than one dose (e.g. two, three, or more than three doses). In particular embodiments, a greater amount of CBD is included in the minor cannabinoid composition to counteract psychoactivity or undesired side effects of THC or the minor cannabinoids.

In certain embodiments, the minor cannabinoid composition is used to deliver about 20, 30, 40, 50, 60, 70, 80, 90, 100, 110, 120, 130, 140, 150, 160, 170, 180, or more than 180 mg/day of THC and about 100, 200, 300, 400, 500, 600, 700, 800, 900, 1000, 1100, 1200, 1300, 1400, 1500, 1600, 1700, 1800, 1900, 2000, 2100, 2200, 2300, or 2400 mg/day of CBD. These amounts may be contained in a single dose, or more than one dose (e.g. two, three, or more than three doses).

In some embodiments, the minor cannabinoid composition is used to deliver about 20, 30, 40, 50, 60, 70, 80, 90, 100, 110, 120, 130, 140, 150, 160, 170, 180, or more than 180 mg/day of the minor cannabinoid. These amounts may be contained in a single dose, or more than one dose (e.g. two, three, or more than three doses). Compositions disclosed herein comprising a minor cannabinoid may further be used to deliver about 20, 30, 40, 50, 60, 70, 80, 90,100, 110, 120, 130, 140, 150, 160, 170, 180, or more than 180 mg/day of THC, or about 100, 200, 300, 400, 500, 600, 700, 800, 900, 1000, 1100, 1200, 1300, 1400, 1500, 1600, 1700, 1800, 1900, 2000, 2100, 2200, 2300, or 2400 mg/day of CBD, or a combination thereof.

### II. THC and CBD Compositions

A THC and CBD composition comprises THC and CBD. Specifically, the THC and CBD composition comprises THC and an amount of CBD to reduce any unwanted side effects of THC or risk factors associated with THC, or increase efficacy of THC or of the composition. Non-limiting examples of side effects or risk factors include psychoactivity, psychosis, anxiety, paranoia, dizziness, confusion, cardiovascular risk, memory impairment, or combinations thereof. In some embodiments, the THC and CBD composition further comprises at least one minor cannabinoid, a terpenoid, an additional compound, or a combination thereof.

As used herein, THC refers to tetrahydrocannabinol, whether synthetically produced or isolated from a plant. THC may refer to (-)-trans-Δ⁹-tetrahydrocannabinol or isomers thereof. As used herein, CBD refers to cannabidiol or isomers thereof, whether synthetically produced or isolated from a plant.

The THC and CBD composition may comprise from about 0.1% to about 99% by weight of THC. The THC and CBD composition may comprise about 5, 10, 15, 20, 25, 30, 35, 40, 45, 50, 55, 60, 65, 70, 75, 80, 85, 90, or 95% of THC. In some embodiments, the THC and CBD composition comprises from about 0.1 to about 0.5, about 0.5 to about 1, about 1 to about 2, about 2 to about 5, about 5 to about 10, about 5 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 99% of THC.

The THC and CBD composition may comprise from about 1% to about 99.9% by weight of CBD. The composition may comprise about 5, 10, 15, 20, 25, 30, 35, 40, 45, 50, 55, 60, 65, 70, 75, 80, 85, 90, 95, 98, 98.5, 99, 99.5, 99.7, or 99.9% of CBD. In some embodiments, the THC and CBD composition comprises from about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, about 80 to about 95, about 95 to about 98, about 98 to about 99.9% of CBD.

In certain embodiments, the THC and CBD composition comprises about 80% CBD to about 99.9% CBD, and about 0.1% THC to about 20% THC. In particular embodiments, the THC and CBD composition comprises about 90% CBD to about 97% CBD and about 3% THC to about 10% THC. In other embodiments, the THC and CBD composition comprises at least about 90% CBD and no more than about 10% THC. For instance, the THC and CBD composition may comprise at least about 90, 91, 92, 93, 94, 95, 96, 97, or 98% CBD and no more than about 10, 9, 8, 7, 6, 5, 4, 3, or 2% THC. In other embodiments, the THC and CBD composition comprises at least about 95% CBD and no more than about 5% THC. For instance, the THC and CBD composition may comprise at least about 95, 96, 97, 98.5, 99, 99.5, or 99.9% CBD and no more than about 5, 4, 3, 2, 1.5, 1, 0.5, or 0.1% THC. In some embodiments, the THC and CBD composition comprises a ratio CBD to THC of about 10:1, 12:1, 15:1, 18:1, 20:1, 22:1, 25:1, 28:1, 30:1, 32:1, 35:1, 38:1, 40:1, 50:1, 75:1, 90:1, 100:1, 110:1, 125:1, 150:1, 200:1, 250:1, 300:1, 400:1, or 500:1. In particular, the CBD: THC ratio may be 100:1.

CBD in a composition of the disclosure may reduce the psychoactive side effects or risk factors of THC or other cannabinoids as measured by levels of prolactin, c-Fox expression, anandamide (AEA) in serum and/or cerebral spinal fluid (CSF), GABA, glutamate, FAAH, ethanolamide (palmitoylethanolamide and oleoylethanolamide), FLAT (FAAH-like anandamide transporter), or other fatty acid binding proteins (FABPs).

CBD may exert its antipsychotic effects as measured by acting as an antagonist, agonist, or inverse agonist to receptors CB1, CB2, and TRPV1 in the prefrontal cortex, amygdala and/or hippocampus, GABAergic neurons in the nucleus accumbens, 5HT-1A, D2, PPARγ (peroxisome proliferator activated receptor gamma), or by interacting with ion channels or enzymes, as described herein, contributing to its anti-inflammatory, anti-oxidative, and/or neuroprotective properties.

CBD's anti-psychotic effects may be measured in the striatum and temporal cortex, ventral tegmental area (VTA), nucleus accumbens, ventral pallidum, mediodorsal thalamic nucleus, prefrontal cortex, anterior cingulate, parahippocampal gyrus, amygdala, right posterior temporal gyrus and right temporal lobe, middle occipital gyrus, cerebellum, left caudate, or dopaminergic mesolimbic pathway.

CBD's anti-psychotic effects may be measured by reduction of psychotomimetic symptoms, amorphine-induced stereotyped behavior, amphetamine, D-amphetamine, and ketamine induced hyperlocomotion, social withdrawal in animal models, reduced scores of Brief Psychiatric Rating Scale (BPRS), Parkinson Psychosis Questionnaire, Positive and Negative Syndrome Scale (PANSS), various other behavioral and neurochemical techniques in animal models, such as apomorphine stereotypy, catalepsy, MK-801 Prepulse inhibition (PPI), hyperlocomotion, or social withdrawal, c-Fos immunohistochemistry, or other techniques.

Methods of isolating, synthesizing, or purifying THC and CBD are known in the art.

### (A) Minor Cannabinoids

The THC and CBD composition may further comprise one or more of the minor cannabinoids disclosed herein. In one embodiment, the THC and CBD composition comprises two or more, three or more, four or more, five or more, six or more, seven or more, eight or more, nine or more, or ten or more of the minor cannabinoids. In yet another embodiment, the THC and CBD composition comprises at least 11, at least 12, at least 13, at least 14, at least 15, at least 16, at least 17, at least 18, at least 19, at least 20, at least 21 or at least 22 or more of the minor cannabinoids.

In some embodiments, the minor cannabinoid is one or more of CBG, CBGA, CBN, CBNA, THCV, THCA, CBC, CBCA, CBDV, and D8-THC. In certain embodiments, the THC and CBD composition comprises at least two, three, four, five, six, seven, eight, nine or ten of the minor cannabinoids selected from the group consisting of CBG, CBGA, CBN, CBNA, THCV, THCA, CBC, CBCA, CBDV, and D8-THC. In particular embodiments, the THC and CBD composition comprises any combination of CBG, CBGA, CBN, CBNA, THCV, THCA, CBC, CBCA, CBDV, and D8-THC.

The THC and CBD composition may comprise from about 1% to about 99.9% by weight of the one or more minor cannabinoids. For instance, the THC and CBD composition may comprise about 0.1, 0.5, 1.5, 2, 2.5, 3, 4, 5, 10, 15, 20, 25, 30, 35, 40, 45, 50, 55, 60, 65, 70, 75, 80, 85, 90, 95, 96, 97, 98, 99, 99.5, 99.7, 99.9% of the one or more minor cannabinoids. In some embodiments, the THC and CBD composition comprises from about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 99% of the one or more minor cannabinoids.

In some embodiments, the THC and CBD composition comprises a ratio of minor cannabinoid to THC of about 5:1, 8:1, 10:1, 12:1, 15:1, 18:1, 20:1, 22:1, 25:1, 28:1, 30:1, 32:1, 35:1, 38:1, 40:1, 50:1, 75:1, 90:1, 100:1, 110:1, 125:1, 150:1, 200:1, 250:1, 400:1, or 500:1. In other embodiments, the THC and CBD composition comprises a ratio of minor cannabinoid to D8-THC of about 5:1, 8:1, 10:1, 12:1, 15:1, 18:1, 20:1, 22:1, 25:1, 28:1, 30:1, 32:1, 35:1, 38:1, 40:1, 50:1, 75:1, 90:1, 100:1, 110:1, 125:1, 150:1, 200:1, 250:1, 400:1, or 500:1. In still other embodiments the THC and CBD composition comprises a ratio of minor cannabinoid to THCV of about 5:1, 8:1, 10:1, 12:1, 15:1, 18:1, 20:1, 22:1, 25:1, 28:1, 30:1, 32:1, 35:1, 38:1, 40:1, 50:1, 75:1, 90:1, 100:1, 110:1, 125:1, 150:1, 200:1, 250:1, 400:1, or 500:1. In particular embodiments, the THC and CBD composition comprises a ratio of CBC, CBL, CBN, CBG, CBDV, and/or CBC to THC, D8-THC, or THCV of about 5:1, 8:1, 10:1, 12:1, 15:1, 18:1, 20:1, 22:1, 25:1, 28:1, 30:1, 32:1, 35:1, 38:1, 40:1 50:1, 75:1, 90:1, 100:1, 110:1, 125:1, 150:1, 200:1, 250:1, 400:1, or 500:1.

In particular embodiments, the THC and CBD composition comprises a ratio of D8-THC to D9-THC of about 1:1, 2:1, 3:1, 4:1, 5:1, 6:1, 7:1, 8:1, 9:1, or 10:1. In other embodiments, the THC and CBD composition comprises a ratio of THCV to D9-THC of about 1:1, 2:1, 3:1, 4:1, 5:1 6:1, 7:1, 8:1, 9:1, or 10:1.

### (B) Terpenoids

In certain embodiments, the THC and CBD composition comprises one or more terpenoids disclosed herein. The THC and CBD composition may comprise from 0% to about 99.9% by weight of the one or more terpenoids. For instance, the THC and CBD composition may comprise about 5, 10, 15, 20, 25, 30, 35, 40, 45, 50, 55, 60, 65, 70, 75, 80, 85, 90, 95, 97, 98, 99, 99.5, or 99, % of the one or more terpenoids. In some embodiments, the THC and CBD composition comprises from about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 90, about 90 to 95, about 95 to 99.9% of the one or more terpenoids.

### (C) Other Compounds

In certain embodiments, the THC and CBD composition comprises one or more additional compounds disclosed herein. In some embodiments, the THC and CBD composition comprises one, two, three, four, five, or more than five of the additional compounds.

The THC and CBD composition may comprise from 0% to about 99% by weight of the one or more additional compounds. For instance, the composition may comprise about 5, 10, 15, 20, 25, 30, 35, 40, 45, 50, 55, 60, 65, 70, 75, or 80% of the one or more additional compounds. In some embodiments, the THC and CBD composition comprises about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, or about 60 to about 80, about 65 to about 85, about 70 to about 90, about 75 to about 95, or about 80 to about 99% of the one or more additional compounds.

### (D) Amounts

The THC and CBD composition may be used to deliver about 20, 30, 40, 50, 60, 70, 80, 90, 100, 110, 120, 130, 140, 150, 160, 170, 180, or more than 180 mg/day of THC. These amounts may be contained in a single dose, or more than one dose (e.g. two, three, or more than three doses).

The THC and CBD composition may further be used to deliver about 100, 200, 300, 400, 500, 600, 700, 800, 900, 1000, 1100, 1200, 1300, 1400, 1500, 1600, 1700, 1800, 1900, 2000, 2100, 2200, 2300, or 2400 mg/day of CBD. These amounts may be contained in a single dose, or more than one dose (e.g. two, three, or more than three doses). In particular embodiments, a greater amount of CBD is included in the composition to reduce undesired side effects of THC or risk factors associated with THC, or increase efficacy of THC. Non-limiting examples of side effects or risk factors include psychoactivity, psychosis, anxiety, paranoia, dizziness, confusion, cardiovascular risk, memory impairment, or combinations thereof.

### III. Pharmaceutical Compositions

The present disclosure also provides pharmaceutical compositions. A pharmaceutical composition may comprise a minor cannabinoid composition disclosed herein or a THC and CBD composition disclosed herein, and at least one pharmaceutically acceptable excipient. The pharmaceutically acceptable excipient may be one or more of a diluent, a binder, a filler, a buffering agent, a pH modifying agent, a disintegrant, a dispersant, a preservative, a lubricant, taste-masking agent, a flavoring agent, and a coloring agent. The amount and types of excipients utilized to form pharmaceutical compositions may be selected according to known principles of pharmaceutical science.

Compositions disclosed herein may optionally comprise one or more additional compounds. Thus, in addition to the therapies described herein, one may also provide to a subject disclosed herein other therapies known to be efficacious for treatment of the disease, disorder, or condition or to reduce side effects. In some embodiments, the additional drug or therapeutically active agent induces anti-inflammatory effects.

### (i) Diluent

The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

### (ii) Binder

Binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

(iii) Filler

Fillers include, but are not limited to, carbohydrates, inorganic compounds, and polyvinylpyrrolidone. By way of non-limiting example, the filler may be calcium sulfate, both di-and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

### (iv) Buffering Agent

Buffering agents include, but are not limited to, phosphates, carbonates, citrates, tris buffers, and buffered saline salts (e.g., Tris buffered saline or phosphate buffered saline).

### (v) pH Modifier

In various embodiments, the excipient may comprise a pH modifier. By way of non-limiting example, the pH modifying agent may be sodium carbonate, sodium bicarbonate, sodium citrate, citric acid, or phosphoric acid.

### (vi) Disintegrant

The disintegrant may be non-effervescent or effervescent. Non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

Dispersants include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

Non-limiting examples of preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

### (ix) Lubricant

Non-limiting examples of lubricants include minerals such as talc or silica; and fats such as vegetable stearin, magnesium stearate, or stearic acid.

### (x) Taste-Masking Agent

Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

### (xi) Flavoring Agent

Flavoring agents may be chosen from synthetic flavor oils and flavoring aromatics and/or natural oils, extracts from plants, leaves, flowers, fruits, and combinations thereof.

(xii) Coloring Agent

Coloring agents include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### IV. Administration

### (a) Dosage Forms

A composition disclosed herein may be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In particular embodiments, the composition may be formulated for sublingual delivery. Sublingual delivery forms (films, tablets, or sprays) are designed to dissolve very rapidly. Examples of such formulations may include ergotamine tartrate, isosorbide dinitrate, isoproterenol HCl, or a combination thereof. The necessary ingredients for the pharmaceutical dosage unit may be processed in accordance with known methods, using or incorporating familiar coatings and additives as required. By way of example only, in addition to the pharmaceutically active components, a dosage unit may contain effective amounts of binders, fillers, disintegrants, sustained-release agents, diluents, anti-adherents, glidants, flow aids, plasticizers and lubricants, which are well known in the field of pharmaceutical processing for sublingual delivery. For instance, the formulation of these tablets may contain, in addition to the active agents, a limited number of soluble excipients, including a binder such as povidone or hydroxypropyl methylcellulose (HPMC), diluents such as lactose, mannitol, starch or cellulose, a disintegrant such as pregelatinized or modified starch, lubricants such as magnesium stearate, stearic acid or hydrogenated vegetable oil, a sweetener such as saccharin or sucrose and suitable flavoring and coloring agents. The process of making sublingual dosage forms may involve, for instance, moistening the blended powder components with an alcohol-water solvent system containing approximately 60% alcohol and 40% water and pressing this mixture into tablets.

In certain embodiments, the composition may be encapsulated in a suitable vehicle to either aid in the delivery of the compound(s) to target cells, to increase the stability of the composition, or to minimize potential toxicity of the composition. As will be appreciated by a skilled artisan, a variety of vehicles are suitable for delivering a composition disclosed herein. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, a composition disclosed herein may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy) propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of active ingredient (e.g. minor cannabinoid, major cannabinoid, terpenoid, combination thereof, etc.),, concentration and composition of lipid, concentration of multivalent cations, rate of mixing, presence of and concentration of solvent.

In another embodiment, a composition disclosed herein may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions.

In yet another embodiment, a composition disclosed herein may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions disclosed herein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

A safe and effective amount of a composition disclosed herein may be an amount that would cause the desired therapeutic effect in a subject while minimizing undesired side effects. In various embodiments, an effective amount of the composition reduces pain associated with chemotherapy.

The amount of a composition described herein that can be combined with a pharmaceutically acceptable carrier to produce a single dosage form will vary depending upon the subject treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions disclosed herein will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a composition of disclosed herein can occur as a single event or over a time course of treatment. For example, the composition may be administered daily, weekly, bi-weekly, or monthly. For treatment of acute conditions, the time course of treatment may be from at least one day to at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities.

### (b) Purity

Generally speaking, a compound included in a composition of the disclosure, such as a minor cannabinoid or a major cannabinoid should be of high purity and consistent quality, regardless of source. For instance, a minor or major cannabinoid used in a composition of the disclosure is at least 90, 91, 92, 93, 94, 95, 96, 97, 98 or 99% pure. In certain embodiments, a minor or major cannabinoid used in a composition of the disclosure is at least 95, 96, 97, 98, or 99% pure. In preferred embodiments, a minor or major cannabinoid used in a composition of the disclosure is at least 97% pure.

Manufacture of a compound for a composition of the disclosure is preferred to meet GLP requirements. In certain embodiments, manufacture of a compound for a composition of the disclosure meets GMP requirements to ensure consistent quality.

Quantitative/qualitative methods to confirm quality and purity include use of High Performance Liquid Chromatography (HPLC), Ultra Performance Liquid Chromatography (UPLC), Nuclear Magnetic Resonance (NMR) spectroscopy, Gas Chromatography (GC), Thin Layer Chromatography (TLC), and standard methods for testing for contamination of microbiological, heavy metal, pesticide, or other contaminants.

A composition disclosed herein may have at least 5%, 10%, 15%, 20%, 25%, 30%, 40%, 50%, 60%, 70%, 80%, 90%, or 95% fewer side effects as compared to compositions in which the minor and major cannabinoids are not at least 90% pure. Side effects, as used herein, refer to any effects of the present composition other than reducing pain or nausea associated with chemotherapy.

### V. Methods

Provided herein are methods of reducing pain associated with a small fiber neuropathy, which may include pain associated with chemotherapy. The method may comprise administering the minor cannabinoid composition or the THC and CBD composition to a subject in need thereof. In one example, the composition is administered in a pharmaceutically effective amount. Administering the composition may modulate at least one biomarker of central and peripheral sensitization in the subject.

In some embodiments, the at least one biomarker comprises NMDA. In other embodiments, the at least one biomarker comprises glutamate. In still other embodiments, the at least one biomarker comprises one or more of CGRP, FAAH, Substance P, 5-HT, NO, GABA, NGF, serotonin, dopamine, AEA, 2-AG, BDNF, microRNA, NF-L, NF-H, OPN, glycerophospholipids, 1-deoxysphingolipids, 2-hydroxybutyrate, 3-hydroxybutyrate, pyruvate, o-acetyl-carnitine, sphingolipids, glutathione, lysine, and cholesteryl esters lyso-phosphatidylcholines. In some embodiments, biomarkers of peripheral sensitization include changes in levels of these same substances.

In some embodiments, the minor cannabinoid composition comprises one or more phytocannabinoids. As used herein, the term “phytocannabinoid” refers to a cannabinoid isolated from a non-synthetic source.

The THC and CBD composition may reduce chemotherapy pain while reducing unwanted side effects of THC or risk factors associated with THC. Non-limiting examples of side effects or risk factors include psychoactivity, psychosis, anxiety, paranoia, dizziness, confusion, increased heart rate, memory impairment, and combinations thereof. The method may comprise administering a pharmaceutically effective amount of the THC and CBD composition, wherein the at least one biomarker of central and peripheral sensitization is modulated, while unwanted side effects or risk factors of THC are minimized.

The THC and CBD composition, when administered to a subject in need thereof, may modulate biomarkers and behavioral indicators of central and peripheral sensitization, or of neurodegeneration. The THC and CBD composition may modulate biomarkers of central and peripheral sensitization by binding as an agonist, antagonist, or inverse agonist to one or more receptors from the group consisting of CB1, CB2 (in the dorsal horn, spinal cord), TRPV-1, TRPV-2, TRPV-3, TRPV-4, TRPV-8, TRPA-1, TRPM-8, 5HT-1A, 5HT-2A, Type 3 5HT, alpha-2 adrenoceptor, PPAR-gamma, GAPDH, PPAR, GPCR-92 NK1, EP, trkB, mGlu1, mGlu4, mGlu5 mGlu6, mGlu7, mGlu8, FAAH, NMDA and its subunits NR1 and NR2A.

The THC and CBD composition may indirectly modulate 5-HT receptors (including 5HT-1A/1B/1D receptors), suppress glutamatergic release via CB1 receptor mediated inhibition of NMDA, activate/desensitize TRPV receptors to modulate CGRP release and influence vasomotor tone, increase AEA levels in cerebrospinal fluid and in the PAG, or decrease CGRP and NO. In some embodiments, the composition modulates biomarkers of central and peripheral sensitization by binding as an agonist, antagonist, or inverse agonist to one or more receptors from the group consisting of CB1, CB2, 5HT, TRPV1, TRPV2, NMDA and glutamate receptors.

The THC and CBD composition may decrease the activity of enzymes that act as biomarkers, such as cyclooxygenase, lipoxygenase and P450-type enzymes such as CYP1A1, CYP1A2, CYP2B6, CYP2C9, CYP3A4, CYP3A5, CYP2A6, CYP2D6, CYP1B1, and/or CYP3A7. Decreasing the activity of one or more of these enzymes may modulate the anti-inflammatory effects that are implicated in SFNs and in particular CIPN.

The THC and CBD composition may exhibit other forms of anti-nociception by inhibiting NF-kB activity, iNOS, corticotropin releasing factor binding protein, cysteine protease ATGB4, photoreceptor-specific nuclear receptor NR2E3, or diacylglycerol lipase. The THC and CBD composition may modulate levels of NMDA, alpha amino-3-hydroxyl-5-methyl-4-isoxazole-proprionate, and kainate receptor modulated neuro-toxicities via CB1, or suppress glutamatergic release by inhibiting modulation of NMDA, mediated by CB1, or increase endo-opioid pre-curser gene expression involved in pain perception. In some embodiments, the THC and CBD composition affects sensitization by modulating levels of NMDA and/or glutamate.

Non-limiting examples of behavioral indicators include behavioral responses in animal models of SFNs, and in particular CIPN, in humans with SFNs and in particular with CIPN, in animal models of spontaneous nociceptive responses in plantar region, models of associated neurological symptoms, and models of associated symptoms of SFN or CIPN specifically. Tests to non-invasively determine the damage and functional impairment of small nerve fibers include QST to detect and quantify subtle sensory abnormalities, electrical current, laser, or contact heat stimuli to evoke averaged potentials in thinly myelinated and unmyelinated nerve fibers, neurophysiological investigation of the autonomic nervous system such as heart rate variability, sympathetic skin response or quantitative sudomotor axon reflex testing (QSART), corneal confocal microscopy, morphological investigation of the unmyelinated and thinly myelinated nerve fibers with skin punch biopsy, and microneurography to assess single nociceptors in vivo and detect spontaneous activity in C-fibers.

1. A method of reducing pain associated with a small fiber neuropathy in a subject in need thereof, comprising administering to the subject a composition comprising a pharmaceutically effective amount of one or more minor cannabinoids alone or in combination with one or more of tetrahydrocannabinol (THC) and a cannabidiol (CBD).

2. The method of claim 1, wherein the small fiber neuropathy is a chemotherapy-induced peripheral neuropathy.

3. The method of claim 1, wherein at least one biomarker of central and peripheral sensitization is modulated in the subject.

4. The method of claim 2, wherein the chemotherapy is selected from the group consisting of a taxane, paclitaxel, a platinum derivative, oxaliplatin, cisplatin, a vinka alkaloid, vincristine, and bortezomib.

5. The method of claim 1, wherein the composition comprises a ratio of the total amount of the one or more minor cannabinoids to the amount of THC, wherein the ratio maximizes the modulation of at least one biomarker of central and peripheral sensitization.

6. The method of claim 5, wherein the composition comprises CBD.

7. The method of claim 1, wherein the composition comprises a ratio of the total amount of the one or more minor cannabinoids to the amount of CBD, wherein the ratio maximizes the modulation of at least one biomarker of central and peripheral sensitization.

8. The method of claim 7, wherein the composition comprises THC.

9. The method of claim 1, wherein the composition further comprises a terpenoid.

10. The method of claim 1, wherein the composition further comprises an additional analgesic agent.

11. The method of claim 3, wherein the biomarker is selected from the group consisting of NMDA, glutamate, CGRP, FAAH, Substance P, 5-HT, NO, GABA, NGF, serotonin, dopamine, AEA, 2-AG, BDNF, microRNA, NF-L, NF-H, OPN, glycerophospholipids, 1-deoxysphingolipids, 2-hydroxybutyrate, 3-hydroxybutyrate, pyruvate, o-acetyl-carnitine, sphingolipids, glutathione, lysine, and cholesteryl esters lyso-phosphatidylcholines.

12. The method of claim 1, wherein the one or more minor cannabinoids are selected from the group consisting of cannabigerovarin (CBG), cannbigerolic acid (CBGA), cannabinol (CBN), cannabinolic acid (CBNA), tetrahydrocannabivarin (THCV), tetrahydrocannabinolic acid (THCA), cannabichromene (CBC), cannabichromenic acid (CBCA), cannabivarin (CBDV), and delta-8-tetrahydrocannabinol (D8-THC).

13. The method of claim 12, wherein composition comprises THC and CBD.

14. A method of reducing pain associated with a small fiber neuropathy in a subject in need thereof, comprising administering to the subject a composition comprising a pharmaceutically effective amount of THC and CBD, wherein at least one biomarker of central and peripheral sensitization or neuronal degeneration is modulated in the subject, while reducing undesired side effects or risk factors of THC.

15. The method of claim 14, wherein the pain associated with the small fiber neuropathy is chemotherapy pain.

16. The method of claim 14, wherein the composition comprises a ratio of the amount CBD to the amount of THC, wherein the ratio maximizes the modulation of at least one biomarker of central and peripheral sensitization while reducing undesired side effects or risk factors of THC.

17. The method of claim 16, wherein the CBD: THC ratio is 30:1 to 300:1.

18. The method of claim 17, wherein the CBD: THC ratio is 100:1.

19. The method of claim 17, wherein the CBD: THC ratio is 30:1.

20. The method of claim 14, wherein the composition further comprises a minor cannabinoid selected from the group consisting of CBG, CBGA, CBN, CBNA, THCV, THCA, CBC, CBCA, CBDV, and D8-THC.

21. The method of claim 14, wherein the composition further comprises a terpenoid.

22. The method of claim 14, wherein the composition further comprises an additional analgesic agent.
